(19) United States
(12) Patent Application Publication
(10) Pub. No.: US 2020/0384145 A1
(43) Pub. Date: Dec. 10, 2020

(54) GAS TREATMENT SYSTEM AND METHOD

(71) Applicant: SUEZ GROUPE, PARIS LA DEFENSE CEDEX (FR)

(72) Inventors: Valérie NASTASI, VILLEBON-SUR-YVETTE (FR); Catherine GRACIAN, Puyloubier (FR)

(21) Appl. No.: 16/971,583

(22) PCT Filed: Feb. 21, 2019
(86) PCT No.: PCT/EP2019/054323

§ 371 Date: Aug. 20, 2020

(30) Foreign Application Priority Data
Feb. 21, 2018 (FR) 1851488

(51) Int. Cl. A61L 9/14; B01D 53/18; B01D 53/14; B01D 53/04

(52) U.S. Cl. CPC A61L 9/145; B01D 53/18; B01D 53/1487; B01D 53/04; B01D 2257/708; A61L 2209/22; B01D 2252/103; B01D 2253/102; A61L 2209/14

(57) ABSTRACT

Disclosed is a process for treating a stale gas, involving the following steps: circulation of the stale gas in a gas/liquid contactor, called a contactor, and then circulation of the stale gas in a filter unit, called a filter unit, the process involving, in the contactor, placing a circulating liquid in contact with the stale gas, the circulating liquid having a temperature of between 2 and 15° C., preferably between 5 and 10° C., and also to a treatment unit for performing the process including a gas/liquid contactor and a filter unit.

### TECHNICAL FIELD

The present invention relates to the field of treating gases such as air and biogas, and especially to the treatment of stale air.

The present invention relates in particular to the removal of odors and odorous Volatile Organic Compounds (VOCs) from stale gas.

The present invention also relates to the pretreatment of biogas.

### PRIOR ART

Biofiltration processes are known in the prior art. These processes require maintenance and control of the operating conditions. Furthermore, the flow of air to be treated must be continuous and constant.

Gas scrubbing processes and the associated purifiers are also known. These processes and devices may be wastewater-based and, in this case, the water consumption is substantial and the odor removal yields relatively limited. There are also gas scrubbers using organic solvents, which present better removal yields for hydrophobic VOCs. However, the use of organic solvents makes the processes more expensive and complex. Furthermore, the use of organic solvents requires increased control of the treatment process. There are also chemical scrubbers, which have very high removal yields on some specific odorous compounds, but are virtually ineffective on the majority of VOCs.

The use of adsorbent filters for treating stale air is also known. Filters require frequent replacement of the filtering medium, which makes this process very expensive.

Thermal oxidation processes are also known in the prior art. These processes require heavy devices requiring regular maintenance and high maintenance costs.

The publication by Thakur Prabhat Kumar et al., Research Journal of Chemical Sciences, 2011, volume 1, pages 83-92 is known in particular in the prior art. Said publication is a scientific review describing the use of biofilters for treating VOCs. It mainly describes the advantages and limitations of biofilters, the difficulties in adapting large-scale biofilters and the parameters to be adjusted to optimize the functioning of biofilters.

In addition, the publication by Faisal I. Khan and Aloke Kr. Ghoshal, Journal of Loss Prevention in the Process Industries, 2000, volume 13, pages 527-545, is known in particular in the prior art. Said publication is a scientific journal providing an overview of VOC treatment processes and also the advantages, drawbacks and implementation conditions thereof. Two categories of processes are described, including processes for destroying VOCs by biofiltration or oxidation and processes for recovering VOCs by adsorption, absorption, condensation or separation.

One aim of the invention is notably to overcome the drawbacks of the processes and devices of the prior art.

An aim of the invention is, in particular:

to improve the yield for the removal of VOCs and odors, in particular, but not exclusively, odorous VOCs, contained in stale air, and/or

to reduce the consumption of raw materials, and/or

to allow the treatment of stale gas (notably stale air and biogas) bearing high concentrations of VOCs, and/or

to facilitate the running and maintenance, and/or

to reduce the energy costs.

Another aim of the invention is to provide a process allowing the additional removal:

of odors, other than those of odorous VOCs, and/or

of particles, and/or

of dusts.

### DESCRIPTION OF THE INVENTION

In order to meet at least one of the abovementioned objectives, a process is proposed for treating a stale gas, involving the following steps, preferably performed in this order:

circulation of the stale gas in a gas/liquid contactor, called a contactor, and then

circulation of the stale gas in a filter unit (notably by adsorption), called a filter unit.

Thus, the process according to the invention involves an absorption step, commonly called a scrubbing step, followed by an adsorption step, called a gas filtration step, whereas the known processes described previously include a particle filtration step, followed by a humidification step and then a biological treatment step (or biofiltration).

For the purposes of the present invention, the term “stale gas” means a gas contaminated with pollutants such as VOCs and/or odorous molecules and/or dusts. They may in particular be hydrophilic VOCs, and notably odorous VOCs. The stale gas may in particular be biogas—i.e. methane resulting from the fermentation of sludge and/or waste, contaminated in particular with pollutants as defined above. In this case, the process of the invention in particular enables purification of the biogas, i.e. raising of its methane (CH₄) concentration.

In the present patent application, stale air may be defined as being polluted air, i.e. air charged with pollutants, for instance, but not exclusively, VOCs (notably odorous VOCs), dusts or odors. It is air resulting, for example, but not exclusively, from an industrial process.

The process according to the invention is characterized in that it involves, in the contactor, placing a circulating liquid in contact with the stale gas, said circulating liquid having a temperature of between 2 and 15° C., preferably between 5 and 10° C.

The gas (notably air) is no longer qualified as stale once it has been subjected to the process according to the invention.

The gas (notably air) is considered to have been treated once it has been subjected to the process according to the invention, i.e. once it meets the emissions standards.

In the present patent application, in the absence of precision, the term “contactor” means a gas/liquid contactor.

A gas/liquid contactor is a means well known to a person skilled in the art who is seeking to recover one or more compounds contained in a gas by means of a liquid which will subsequently be recovered. Thus, the gas/liquid contactor makes it possible to extract one or more compounds contained in a gas by performing a mass transfer from a gaseous phase, i.e. the stale gas, to a liquid phase, i.e. the circulating liquid. In practice, the functioning of a contactor typically consists in:

placing a liquid in contact with a gas comprising one or more compounds that it is sought to recover so that optimal transfer of the compound(s) from the gas to the liquid takes place, and then

recovering the liquid containing the compound(s).

The gas/liquid contactor may contain packing, aimed at increasing the exchange surface between the stale gas and the circulating liquid, to promote the mass transfer (or scrubbing). However, the gas/liquid contactor does not contain any adsorbent solid support designed so that pollutants such as VOCs and/or odorous molecules and/or dusts contained in the stale gas are adsorbed onto its surface. In particular, the gas/liquid contactor does not contain any active charcoal.

According to the invention, the step of circulating the stale gas in the gas/liquid contactor is directed toward reducing the amount of pollutants such as VOCs and/or odorous molecules and/or dusts contained in the stale gas by transfer into the circulating liquid. Thus, this step of the process is likened to a step of physicochemical scrubbing of the stale gas using the circulating liquid. The phenomenon involved is thus typically absorption.

One of the advantages of the process according to the invention is that it does not include a step of biological treatment (biofiltration) of the stale gas.

For the purposes of the invention, the term “filter unit” means a filter unit based on purely physical phenomena such as adsorption (physisorption). Thus, there is no chemical transformation of the molecules retained by the filter unit; it is therefore not a unit for destroying VOCs or pollutants, nor a biological unit.

The liquid circulating in the contactor may be water, oil or an organic solvent.

Preferably, the liquid circulating in the contactor may be water, notably industrial water.

The industrial water may be filtered; preferably, the industrial water may be filtered between 25 and 750 μm, more preferably between 150 and 350 μm.

The liquid circulating in the contactor may be water coming from a refrigerating device designed to cool water.

In the case where the liquid circulating in the contactor comes from a refrigerating device designed to cool water, the refrigerating device may be fed with industrial water.

A flow rate of the liquid circulating in the contactor relative to a flow rate of the stale air circulating in the contactor may be less than 20 l/m³, preferably less than 10 l/m³. This flow rate is commonly referred to by those skilled in the art as the liquid-to-gas ratio.

According to the invention, the circulation of the stale gas (notably the air) in the contactor may involve circulation of the stale gas (notably the air) in a direction opposite to a direction in which the liquid circulates in the contactor.

Thus, in accordance with the first aspect of the invention, the circulation of the stale air in the contactor may involve:

circulation of the stale gas in a direction identical to a direction in which the liquid circulates in the contactor, and

circulation of the stale gas in a direction opposite to a direction in which the liquid circulates in the contactor.

Preferably, the step of circulating the stale gas in a direction identical to a direction in which the liquid circulates in the contactor is performed prior to the step of circulating the stale gas in a direction opposite to a direction in which the liquid circulates in the contactor.

The process according to the invention may involve:

injection of the stale gas (notably the air) into a first part of the contactor, called the co-current part, in which the stale gas circulates in the same direction as the direction in which the liquid circulates in said first part of the contactor, and

circulation of the stale gas (notably the air) in a second part of the contactor, called the counter-current part, in which the stale gas circulates in the direction opposite to the direction in which the liquid circulates in said second part of the contactor, and then

circulation of the stale gas in the filter unit.

Thus, in other words, the process may involve:

circulation of the stale gas in co-current with the liquid circulating in the contactor, the stale gas circulates in the same direction as the direction in which the liquid circulates in said first part, and then

circulation of the stale gas in counter-current to the liquid circulating in the contactor, the stale gas circulates in the opposite direction to the direction in which the liquid circulates in said second part, and then

circulation of the stale gas in the filter unit.

Preferably, the stale gas (notably air) injected into the contactor may have a temperature of greater than 5° C., notably between 5 and 80° C.

More preferably, the stale gas injected into the contactor may have a temperature between 15 and 60° C., even more preferably between 35 and 55° C.

According to a first embodiment, the stale gas (in particular air) injected into the contactor may have a temperature of between 40 and 50° C. According to another embodiment, the stale air injected into the contactor may have a temperature of between 25 and 35° C.

According to the invention, the process may involve placing the stale gas (notably air) and the liquid circulating in the contactor in contact with a heat exchanger.

The step of placing the stale gas and the liquid circulating in the contactor in contact with the heat exchanger may be performed, totally or partly, concomitantly with the step of circulating the stale gas in co-current with the liquid circulating in the contactor, in other words, in the first part of the contactor.

Preferably, the process may involve circulation of a cooling liquid in the heat exchanger, said cooling liquid having a temperature of between 2 and 15° C., preferably between 3 and 10° C.

More preferably, the cooling liquid may have a temperature of 5° C.

Even more preferably, the temperature of the cooling liquid may be equal to the temperature of the liquid circulating in the contactor.

The cooling liquid may be recovered for:

reinjection into a cooling liquid circuit, and/or

reinjection into the contactor, and/or

reinjection into a circuit for the liquid circulating in the contactor.

The cooling liquid may be water.

Preferably, the cooling liquid may be industrial water.

The industrial water may be filtered; preferably, the industrial water may be filtered between 25 and 750 μm, more preferably between 150 and 350 μm.

The cooling liquid may be water coming from a refrigerating device designed to cool water.

The cooling liquid may be water coming from a refrigerating device designed to cool water.

In the case where the liquid circulating in the contactor comes from a refrigerating device designed to cool water, the refrigerating device may be fed with industrial water.

Alternatively, the cooling liquid may be a refrigerant liquid, such as glycol, ethylene glycol or monoethylene glycol (MEG), preferably MEG.

According to the invention, the process may involve:

injection of the liquid circulating in the contactor into the second part of the contactor or, respectively, into the first part of the contactor, and then

reinjection of the liquid which has circulated in the second part of the contactor into the first part of the contactor or, respectively, into the second part of the contactor.

Preferably, the process may involve:

injection of the liquid circulating in the contactor into the second part of the contactor, and then

reinjection of the liquid which has circulated in the second part of the contactor into the first part of the contactor.

Subsequent to the circulation, in the first part of the contactor, of the reinjected liquid or, respectively, in the second part of the contactor, the process may involve recovery of the liquid which has circulated in the contactor.

The recovered liquid, which has circulated in the contactor, can be removed for the purpose of its subsequent treatment and/or recycling.

The process may include a step of heating the stale gas (notably the air) prior to the circulation of the stale gas in the filter unit.

Preferably, the step of heating the stale gas is performed subsequent to the step of circulating the stale gas in the contactor.

During this heating step, the stale gas may be heated to a temperature above 3° C., notably between 5 and 35° C., preferably between 10 and 30° C.

During this heating step, in a particularly preferred manner, the stale gas may be heated to a temperature 5° C. higher than its temperature at the outlet of the contactor.

Preferably, the circulation of the stale gas in the first part of the contactor may take place in a downward or, respectively, upward swirling movement, around a central zone of the contactor and the circulation of the stale gas in the second part of the contactor may take place in a substantially rectilinear upward or, respectively, downward movement in the central zone of the contactor.

In accordance with the invention, the circulation of stale gas may take place at a flow rate of between 100 and 20 000 m³/h, preferably between 250 and 10 000 m³/h, more preferably between 500 and 5000 m³/h.

In other words, between 100 and 20 000 m³, preferably between 250 and 10 000 m³, more preferably between 500 and 5000 m³ are injected into the contactor and are recovered at the contactor outlet per hour.

The process according to the invention may be used for removing odorous Volatile Organic Compounds (VOCs).

The process may also be used for removing:

hydrophilic VOCs, and/or

odorous hydrophilic VOCs, and/or

dust, and/or

particles, and/or

odorous compounds other than VOCs, and/or

nonodorous VOCs.

Preferably, the process is used for treating stale air, notably originating from:

sludge, and/or

treatment of sludge derived from water treatment, and/or

wastewater treatment processes, and/or

waste treatment facilities.

It is possible to use the process according to the invention for treating gases, in particular stale air containing VOCs present in concentrations of greater than 10 mg/m³, preferably between 10 and 1000 mg/m³, more preferably between 50 and 500 mg/m³.

According to the invention, a unit for treating stale gas (notably air) is also proposed, comprising:

a gas/liquid contactor, called a contactor, in which the stale gas circulates,

a filter unit, called a filter unit, in which the stale gas circulates; said stale gas treatment unit being characterized in that it is designed to perform the process according to the first aspect of the invention.

The gas (notably air) is no longer qualified as stale once it has passed through the treatment unit.

The gas, in particular air, is considered as treated once it has passed through the treatment unit as described according to the second aspect of the invention.

Preferably, the treatment unit according to is designed to perform the process according to the invention.

In accordance with the invention, the filter unit may be any device known to those skilled in the art and is preferably an active charcoal filter unit.

In accordance with the invention, the treatment unit may comprise one or more contactors.

The treatment unit is arranged so that a flow rate of the liquid circulating in the contactor relative to a flow rate of the stale gas circulating in the contactor may be less than 20 l/m³, preferably less than 10 l/m³.

The unit for treating stale gas (notably air) may be arranged so that a liquid circulating in the contactor is injected into a second part of the contactor or, respectively, into a first part of the contactor, and so that the liquid which has been injected into the second part of the contactor is reinjected into the first part of the contactor or, respectively, into the second part of the contactor,

said liquid circulating in the contactor having a temperature of between 2 and 15° C., preferably between 5 and 10° C.

The gas/liquid contactor is arranged so that the liquid circulating in the contactor comes into direct contact with the stale gas circulating in said contactor.

Preferably, the stale gas injected into the contactor may have a temperature of greater than 5° C., notably between 5 and 80° C.

More preferably, the stale gas injected into the contactor may have a temperature of between 15 and 60° C., even more preferably between 35 and 55° C.

Particularly preferably, the stale gas injected into the contactor may have a temperature of between 40 and 50° C.

The liquid circulating in the contactor may be water, oil or an organic solvent. Preferably, the circulating liquid is water, notably industrial water.

The first part of the contactor, called the co-current part, may be arranged so that the stale gas circulates in the same direction as a direction in which the liquid circulating in the contactor circulates in said first part of the contactor, and the second part of the contactor, called the counter-current part, may be arranged so that the stale gas circulates in a direction opposite to a direction in which the liquid circulating in the contactor circulates in said second part of the contactor.

Preferably, the second part of the contactor may be a central zone of the contactor in which the circulation of the stale gas can take place in a substantially rectilinear upward or, respectively, downward movement, and the first part of the contactor may be a peripheral zone of the contactor in which the circulation of the stale gas can take place in a downward or, respectively, upward swirling movement around the first part.

Preferably, the central zone can extend along a central axis of the contactor. For example, the central axis may be an axis of revolution of the contactor.

The second part of the contactor may extend from an outer edge of the central zone to an inner edge of the contactor.

The unit for treating stale gas (notably air) may include a heat exchanger arranged so that the stale gas and the liquid circulating in the contactor come into contact with an exchange surface of the exchanger inside which circulates a cooling liquid whose temperature is between 2 and 15° C., preferably between 5 and 10° C.

The cooling liquid may be water or a refrigerant liquid, as defined above in connection with the process of the invention.

In accordance with the invention, the treatment unit may be arranged so that the stale gas, notably stale air:

circulates in the contactor, and then

circulates in the filter unit.

Preferably, the stale gas treatment unit may be arranged so that the liquid which has circulated in the contactor is recovered for the purpose of its subsequent treatment and/or recycling.

According to the invention, the stale gas treatment unit may include a stale gas heating element arranged so that the stale gas is heated before entering the filter unit.

The heating element may be any heating means known to a person skilled in the art, and may notably comprise a hot filament placed on the circulation path of the stale gas.

In accordance with the invention, the contactor may be any type of contactor known to those skilled in the art. By way of example, the contactor may be of the following type: spray column, packed column, bubble column, plate column, falling-film column or cyclone.

Advantageously, the contactor is a contactor of the “cyclonic exchanger” type.

The unit for treating stale gas, in particular stale air, may be arranged for the removal, inter alia, of Volatile Organic Compounds (VOCs) (in particular odorous VOCs) contained in the stale air, notably stale air containing VOCs in a concentration of greater than 10 mg/m³, preferably between 10 and 1000 mg/m³, more preferably between 10 and 500 mg/m³.

The unit for treating stale gas (notably stale air) may be arranged so that the stale gas circulates at a flow rate of between 100 and 20 000 m³/h, preferably between 2500 and 10 000 m³/h, more preferably between 500 and 5000 m³/h.

The unit for treating stale gas, notably stale air, may include a device for cooling the water injected into the contactor and/or into the heat ex-changer.

The cooling device may be any refrigerating device designed to cool water and/or produce cold, such as, inter alia, gas compression and/or gas absorption systems and/or heat pump systems.

### DESCRIPTION OF THE FIGURES AND EMBODIMENTS

Other advantages and features of the invention will become apparent on reading the detailed description of implementations and embodiments which are in no way limiting, and from the following appended drawings:

FIG. 1 is a schematic representation of a first embodiment of the process and of the treatment unit according to the invention;

FIG. 2 is a schematic representation of a second embodiment of the treatment unit and of the process according to the invention.

Since the embodiments described below are in no way limiting, it is notably possible to consider variants of the invention comprising only a selection of described features, isolated from the other described features (even if this selection is isolated within a sentence including these other features), if this selection of features is sufficient to confer a technical advantage or to differentiate the invention from the prior art. This selection comprises at least one feature, which is preferably functional without structural details, or with only part of the structural details if this part only is sufficient to confer a technical advantage or to differentiate the invention from the prior art.

In addition, the embodiments below are described in relation with the treatment of stale air, but can also be applied to the treatment of biogas.

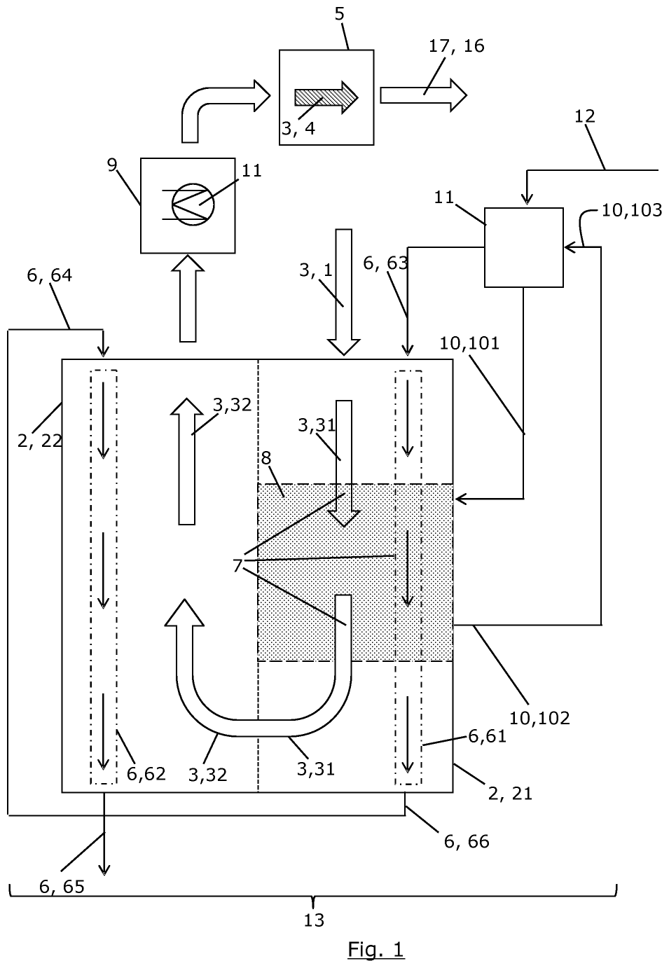

With reference to FIG. 1, a first embodiment describes a treatment process according to the invention and also a treatment unit according to the invention.

FIG. 1 presents a process for treating odors and odorous VOCs of stale air 3 resulting from a process for treating wastewater or waste treatment facilities. The stale air 3 is introduced 1 into the treatment unit 13 at a temperature of between 35° C. and 55° C. The process involves circulation 31, 32 of the stale air 3 in a gas/liquid contactor 2, called the contactor 2, and circulation 4 of the stale air in a filter unit 5, called the filter unit 5. The process involves placing a liquid 6 circulating 61, 62 in the contactor 2 in contact with the stale air 3 circulating 31, 32 in the contactor 2. The circulating 61, 62 liquid 6 has a temperature of between 5 and 10° C. This temperature may, in certain cases, range between 2 and 15° C. The placing of the liquid 6 circulating 61, 62 in the contactor 2 in contact with the stale air 3 circulating 31, 32 in the contactor 2 is direct contact.

The liquid 6 circulating 61, 62 in the contactor 2 is water.

The circulation 31, 32 of the stale air 3 in the contactor 2 involves circulation 32 of the stale air 3 in a direction opposite to a direction 61 in which the liquid 6 circulates in the contactor 2.

The flow rate of the liquid 6 circulating 61, 62 in the contactor 2 relative to the flow of stale air 3 circulating in the contactor 2 is less than 10 l/m³. According to the invention, the flow rate of the liquid 6 circulating 61, 62 in the contactor 2 relative to the flow of stale air 3 circulating in contactor is from 2 to 4 ml/m³, advantageously 3 l/m³.

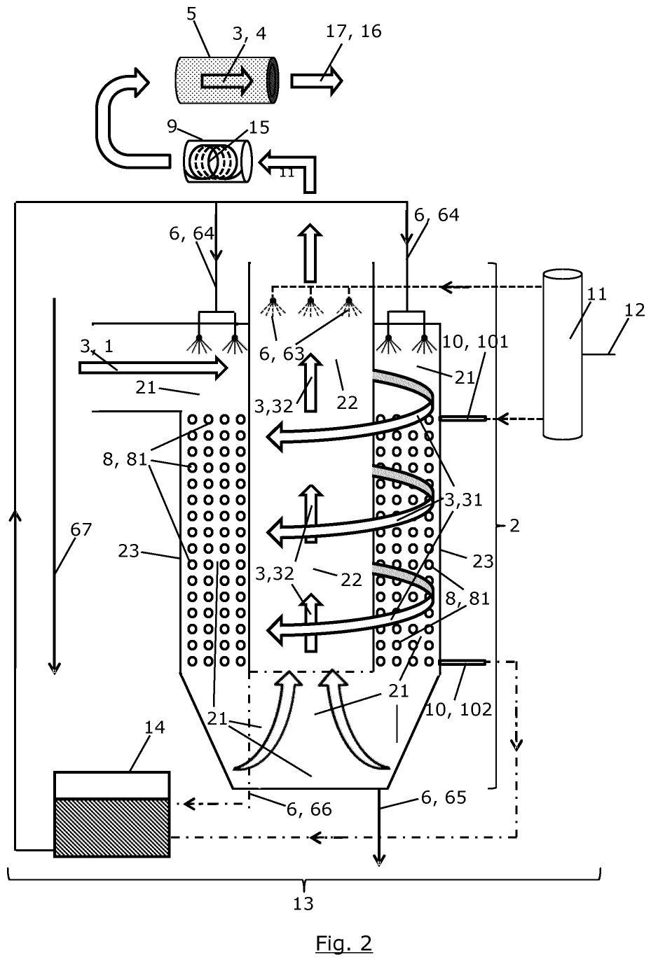

With reference to FIG. 2, the liquid 6 circulating 61, 62 in the contactor 2 circulates in a single vertical direction 67 in which the liquid 6 flows.

The circulation 31, 32 of the stale air 3 in the contactor 2 involves circulation 31 of the stale air 3 in a direction identical to the direction 61 in which the liquid 6 circulates in the contactor 2 and circulation 32 of the air stale 3 in a direction opposite to the direction 62 in which the liquid 6 circulates in the contactor 2. According to, the step of circulation 31 of the stale air 3 in a direction identical to the direction 61 in which the liquid 6 circulates in the contactor 2 is performed prior to the step of circulation 32 of the stale air 3 in a direction opposite to the direction 62 in which the liquid 6 circulates in the contactor 2.

The process according to the invention involves injection 1 of the stale air into a first part 21 of the contactor 2, called the co-current part, in which the stale air 3 circulates 31 in the same direction as the direction in which the liquid 6 circulates 61 in said first part 21 of the contactor 2. Equivalently, this step may be described as circulation 31 of the stale air 3 in co-current with the liquid 6 circulating 61 in the contactor 2. Next, the process involves circulation 32 of the stale air 3 in a second part 22 of the contactor 2, called the counter-current part 22, in which the stale air circulates 32 in the opposite direction to the direction in which the liquid 6 circulates 62 in said second part 22 of the contactor 2. Equivalently, this step may be described as circulation 32 of the stale air 3 in counter-current to the liquid 6 circulating 62 in the contactor 2. Next, the process involves circulation 4 of the stale air 3 in the filter unit 5. At the outlet of the treatment unit 13, the process involves discharging 16 the treated air 17.

Subsequent to the step of circulating 62 stale air in the second part 22 of the contactor 2, the process includes a step of heating 9 the stale air 3 prior to circulation 4 of the stale air 3 in the filter unit 5. The step of heating 9 the stale air 3 is thus performed subsequent to the step of circulating 61 the liquid 6 in the first part 21 of the contactor 2. During the heating step, the stale air 3 is heated to a temperature 5° C. higher than its temperature at the outlet of the contactor 2.

The process involves injection 63, into the first part 21 of the contactor 2, of the liquid 6 circulating 61, 62 in the contactor 2, followed by recovery 66 of the liquid 6 which has circulated 61 in the first part 21 of the contactor 2. The recovery 66 of the liquid 6 is followed by reinjection 64, into the second part 22 of the contactor 2, of the liquid 6 which has circulated 61 in the first part 21 of the contactor 2.

Subsequent to the circulation 62, in the second part 22 of the contactor 2, of the reinjected 64 liquid 6, the process involves recovery 65 of the liquid 6 which has circulated 61, 62 in the contactor 2.

The recovered 65 liquid 6, which has circulated 61, 62 in the contactor 2 is removed for the purpose of its subsequent treatment and/or recycling.

The process involves placing stale air 3 circulating 31, 32 in the contactor 2 and liquid 6 circulating 61, 62 in the contactor 2 in contact 7 with a heat exchanger 8.

The liquid 10 circulating in the heat exchanger, called the cooling liquid 10, is water.

The step of placing the stale air 3 and the liquid 6, circulating 31, 32, 61, 62 in the contactor 2, in contact 7 with the heat exchanger 8 is performed concomitantly with the step of circulating 31 the stale air 3 in co-current with the liquid 6 circulating 61 in the contactor 2. In other words, the step of placing the stale air 3 and the liquid 6 circulating 31, 32, 61, 62 in the contactor 2 in contact 7 with the heat exchanger 8 is performed in the first part 21 of the contactor 2. The cooling liquid 10 circulating in the heat exchanger 8 has a temperature of between 5 and 10° C. This temperature may, in certain cases, range between 2 and 15° C.

The cooling liquid 10 injected 101 into the heat exchanger 8 is identical to the liquid 6 injected 63 into the contactor 2. The cooling liquid 10 injected 101 into the exchanger 8 and the liquid 6 injected 63 into the contactor 2 come from a refrigerating unit 11 fed with industrial water 12. The cooling liquid 10 is recovered 102, at the outlet of the heat exchanger 8, and is reinjected 103 into the cooling liquid 10 circuit. According to the invention, the cooling liquid 10 is reinjected 103 into the refrigerating unit 11.

With reference to FIG. 2, a second embodiment describes a treatment unit according to the invention and also a process according to the invention.

FIG. 2 presents a unit 13 for treating stale air 3. The treatment unit 13 comprises a gas/liquid contactor 2, called the contactor 2, in which the stale air 3 circulates 31, 32 and a filter unit 5 in which the stale air 3 circulates 4. The treatment unit 13 is arranged so that the stale air 3 circulates 31, 32 in the contactor 2 then circulates 4 in the filter unit 5. The stale air 3 is injected 1 into the first part 21 of the contactor 2. The stale air is injected 1 into the contactor 2 at a temperature of between 35 and 55° C. The treated air 16, leaving the treatment unit 13, is discharged 17 into the atmosphere.

The stale air 3 treatment unit 13 is designed to perform the process according to the invention.

According to the invention, the filter unit 5 is an active charcoal filter unit 5.

The stale air 3 treatment unit 13 is arranged so that the liquid 6 circulating 61, 62 in the contactor 2 is injected 63 into a second part 22 of the contactor 2 and so that the liquid 6 which has been injected 63 into the second part 22 of the contactor 2 is reinjected 64 into the first part 21 of contactor 2. The liquid 6 circulating 61, 62 in the contactor 2 has a temperature of between 5 and 10° C. This temperature may, in certain cases, range between 2 and 15° C.

The placing of the liquid 6 circulating 61, 62 in the contactor 2 in contact with the stale air 3 circulating 31, 32 in the contactor 2 is direct contact. The injections 63 and 64 of liquid 6 into the contactor 2 are performed by spraying via nozzles.

The stale air 3 treatment unit 13 is arranged so that the flow rate of the liquid 6 circulating 61, 62 in the contactor 2 relative to the flow rate of stale air 3 circulating in the contactor 2 is less than 10 l/m³. According to the invention, the flow rate of the liquid 6 circulating 61, 62 in the contactor 2 relative to the flow rate of stale air 3 circulating in contactor 2 is between 2 and 4 L/m³, for example 3 l/m³.

The liquid 6 circulating 61, 62 in the contactor 2 is water.

The liquid 6 circulating 61, 62 in the contactor 2 circulates in a single vertical direction 67 in which the liquid 6 flows by gravity.

The first part 21 of the contactor 2, called the co-current part 21, is arranged so that the stale air 3 circulates 31 in the same direction 67 as the direction in which the liquid 6 circulating 61, 62 in the contactor 2 circulates 61 in said first part 21 of the contactor 2. The second part 22 of the contactor 2, called the counter-current part 22, is arranged so that the stale air 3 circulates 32 in the opposite direction to the direction 62 in which the liquid 6 circulating 61, 62 in the contactor 2 circulates 62 in said second part 22 of the contactor 2.

The contactor 2 is a “cyclone” type contactor 2. The circulation 31 of the stale air 3 in the first part 21 of the contactor 2 takes place in a downward swirling movement 31 around the central zone 22 of the contactor 2. The circulation 32 of the stale air 3 in the second part 22 of the contactor 2 takes place in a substantially rectilinear upward movement 32 in the central zone 22 of the contactor 2.

The “cyclone” type contactor 2 has a cylindrical shape. The central zone 22 extends along the axis of revolution of the contactor 2.

The second part 22 of the contactor extends from the outer walls of the central zone 22 to the inner walls 23 of the contactor 22, i.e. the inner walls 23 of the first part 21 of the contactor 2.

The stale air 3 treatment unit 13 comprises a heat exchanger 8 arranged so that the stale air 3 and the liquid 6 circulating 31, 32, 61, 62 in the contactor 2 come into contact with an exchange surface of the exchanger 8. Inside the exchange surface 81 circulates a cooling liquid 10, the temperature of which is between 5 and 10° C. This temperature may, in certain cases, range between 2 and 15° C.

The cooling liquid 10 is water.

The heat exchanger 8 is a tubular exchanger 8. The exchanger 8 comprising a bundle of circular tubes 81 extending around the central zone 22.

The cooling liquid 10 and the liquid 6 circulating 61, 62 in the contactor 2 are recovered, respectively 102 and 66, in a tank 14, then reinjected 64 into the first part 21 of the contactor 2.

The tank 14 forms part of a recirculation circuit.

The cooling liquid 10 injected 101 into the heat exchanger 8 is identical to the liquid 6 injected 63 into the contactor 2. The cooling liquid 10 injected 101 and the liquid 6 injected 63 come from a refrigerating unit 11 fed with industrial water 12. The cooling liquid and the liquid 6 which has circulated 62 in the second part 22 of the contactor 2 are recovered 102, 66 at the outlet of the heat exchanger 8, in a tank 14.

The stale air 3 treatment unit 13 is arranged so that the liquid 6 which has circulated 61, 62 in the contactor 2 is recovered 65 for the purpose of its subsequent treatment. The liquid 6 is recovered 65 subsequent to the circulation 61, in the first part 21 of the contactor 2, of the reinjected 64 liquid 6.

The recovered 65 liquid 6, which has circulated 6, 61, 62 in the contactor 2, is removed for the purpose of its subsequent treatment and/or recycling.

The stale air 3 treatment unit 13 comprises an element 9 for heating the stale air 3, arranged so that the stale air 3 is heated before circulating 4 in the filter unit 5. The stale air 3 is heated to a temperature 5° C. higher than its temperature at the outlet of the contactor 2.

The heating element 3 comprises a hot filament 15 positioned on the path of circulation of the stale air 3 so as to heat the stale air 3.

Although, according to the embodiments presented, the main intended application, for the process and the treatment unit 13, is the treatment of odors and odorous VOCs, the process and the treatment unit 13 according to the invention also perform the removal of:

hydrophilic VOCs, and/or

odorous hydrophilic VOCs, and/or

dusts, and/or

particles, and/or

odorous compounds other than VOCs, and/or

nonodorous VOCs.

Although, according to the embodiments presented, the main intended application, for the process and the treatment unit 13, is the treatment of stale air 3 originating from wastewater or sludge, in particular sludge resulting from water treatment, or from waste treatment facilities. The process and the treatment unit 13 are also suitable for treating any type of stale air 3 containing elements such as mentioned above.

The stale air 3 injected 1 into the contactor 2 has a temperature above 5° C. In general, when the stale air 3 comes from a water treatment process, it has a temperature above 18° C., notably between 40° C. and 50° C.

The process and the treatment unit 13 are suitable for treating stale air 3 containing VOCs present in concentrations of greater than 10 mg/m³, preferably between 10 and 1000 mg/m³, more preferably between 10 and 35 500 mg/m³.

The efficiency of the process and of the treatment unit 13 is such that it allows circulation 31, 32, 4 of stale air 3 at a flow rate of between 100 and 20 000 m³/h. For optimal treatment performance, the circulation 31, 32, 4 of stale air 3 is performed at a flow rate of between 500 and 5000 m³/h. In other words, between 500 and 5000 m³ are injected into the contactor 2 and are recovered at the outlet of the contactor 2, and thus of the filter unit 5, per hour.

Needless to say, the invention is not limited to the examples that have just been described and numerous amendments may be made to these examples without departing from the scope of the invention.

Thus, in variants that may be combined with each other of the embodiments described previously:

the stale air 3 injected 1 into the contactor 2, or into the treatment unit 13, has a temperature above 5° C., and/or

the stale air 3 injected 1 into the contactor 2, or into the treatment unit 13, has a temperature of between 15 and 60° C., and/or

the stale air 3 injected 1 into the contactor 2, or into the treatment unit 13, has a temperature of between 5 and 80° C., and/or

the flow rate of the liquid 6 circulating 61, 62 in the contactor 2 relative to the flow of stale air 3 circulating in the contactor 2 is less than 20 l/m³, and/or

the cooling liquid 10 and/or the liquid 6 circulating 61, 62 in the contactor 2 have a temperature of 5° C., and/or

the temperature of the cooling liquid 10 is equal to the temperature of the liquid 6 circulating 61, 62 in the contactor 2, and/or

the liquid 6 circulating 61, 62 in the contactor 2 and/or the cooling liquid 10 is industrial water filtered at 250 μm, and/or

the process involves:
injection 63 of the liquid 6 circulating 61, 62 in the contactor 2 into the second part 22 of the contactor 2, and then
reinjection 64 of the liquid 6 which has circulated 61 in the second part 22 of the contactor 2 into the first part 21 of the contactor 2, and/or

the process includes a step 9 of heating the stale air 3 prior to the circulation 4 of the stale air 3 in the filter unit 5, and/or

the stale air 3 is heated to a temperature above 3° C., and/or

the stale air 3 is heated to a temperature of between 5 and 35° C., preferably between 10 and 30° C., and/or

the stale air 3 is heated to a temperature of 25° C., and/or

the cooling liquid 10 is recovered 102, at the outlet of the heat exchanger 8, and is reinjected:
into the contactor 2, and/or
into a circuit for the liquid 6 circulating 61, 62 in the contactor 2, and/or

the circulation 31 of the stale air 3 in the first part 21 of the contactor 2 takes place in an ascending swirling movement 31 around the central zone 22 of the contactor 2; the circulation 32 of the stale air 3 in the second part 22 of the contactor 2 takes place in a substantially rectilinear downward movement 32 in the central zone 22 of the contactor 2, and/or

the efficiency of the process and of the treatment unit 13 is such that it allows circulation 31, 32, 4 of stale air 3 at a flow rate of between 250 and 10 000 m³/h,

the unit 13 for treating stale air 3 is arranged so that the liquid 6 circulating 61, 62 in the contactor 2 is injected 63 into a first part 21 of the contactor 2 and so that the liquid 6 which has been injected 63 into the first part 21 of the contactor 2 is reinjected 64 into second part 22 of the contactor 2, and/or

the refrigerating unit 11 may be any device designed to produce cold, such as:
a gas compression system, or
a gas absorption system, or
a heat pump system, or

the cooling liquid 10 and the liquid 6 circulating 61, 62 in the contactor 2 are recovered, respectively 102 and 66, in a tank 14 to be reinjected 64:
into a cooling liquid circuit 10, and/or
into a circuit for the liquid 6 circulating 61, 62 in the contactor 2, and/or
into the second part 22 of the contactor 2, and/or

the contactor is a contactor of the following type:
spray column, or
packed column, or
tray column, or
bubble column, or
falling-film column, and/or

the treatment unit 13 comprises several contactors 2, and/or

when the treatment unit 13 comprises several contactors 2, the contactors 2 are arranged in series.

Furthermore, the various features, forms, variants and embodiments of the invention can be combined with each other in various combinations provided that they are not incompatible or mutually exclusive.

1. A process for treating a stale gas, comprising:
circulating the stale gas in a gas/liquid contactor, called a contactor, and then
circulating the stale gas in a filter unit, called a filter unit, the process including, in the contactor, placing a circulating liquid in contact with the stale gas, said circulating liquid having a temperature of between 2 and 15° C.

2. The process as claimed in claim 1, wherein the circulation of the stale gas in the contactor involves circulation of the stale gas in a direction opposite to a direction in which the liquid circulates in the contactor.

3. The process as claimed in claim 2, wherein the circulation of the stale gas in the contactor involves:
circulating the stale gas in a direction identical to a direction in which the liquid circulates in the contactor,
circulating the stale gas in a direction opposite to a direction in which the liquid circulates in the contactor.

4. The process as claimed in claim 1, further comprising:
injecting the stale gas into a first part of the contactor, called the co-current part, in which the stale gas circulates in the same direction as the direction in which the liquid circulates in said first part of the contactor, and
circulating the stale gas in a second part of the contactor, called the counter-current part, in which the stale gas circulates in the direction opposite to the direction in which the liquid circulates in said second part of the contactor, and
circulating the stale gas in the filter unit.

5. The process as claimed in claim 1, further comprising placing the stale gas and the liquid circulating in the contactor in contact with a heat exchanger.

6. The process as claimed in claim 1, further comprising circulating a cooling liquid in the heat exchanger, said cooling liquid having a temperature of between 2 and 15° C.

7. The process as claimed in claim 1, further comprising:
injecting the liquid circulating in the contactor into the second part of the contactor or, respectively, into the first part of the contactor, and then
reinjecting the liquid which has circulated in the second part of the contactor into the first part of the contactor or, respectively, into the second part of the contactor.

8. The process as claimed in claim 1, further comprising a step of heating the stale gas prior to the circulation of the stale gas in the filter unit.

9. The process as claimed in claim 1, wherein the process is performed for the removal of odorous volatile organic compounds (VOCs).

10. The process as claimed in claim 1, wherein the stale gas is stale air.

11. The process as claimed in claim 1, wherein the stale gas is biogas.

12. A stale gas treatment unit comprising:
a gas/liquid contactor, called a contactor, in which the stale gas circulates,
a filter unit, called a filter unit, in which the stale gas circulates;
said stale gas treatment unit being designed to perform the process as claimed in claim 1.

13. The stale gas treatment unit as claimed in claim 12, wherein the unit is arranged so that a liquid circulating in the contactor is injected into a second part of the contactor or, respectively, into a first part of the contactor, and so that the liquid which has been injected into the second part of the contactor is reinjected into the first part of the contactor or, respectively, into the second part of the contactor;
said liquid circulating in the contactor having a temperature of between 2 and 15° C.

14. The stale gas treatment unit as claimed in claim 12, wherein:
the first part of the contactor, called the co-current part, is arranged so that the stale gas circulates in a same direction as a direction in which the liquid circulating in the contactor circulates in said first part of the contactor, and
the second part of the contactor, called the counter-current part, is arranged so that the stale gas circulates in an opposite direction to a direction in which the liquid circulating in the contactor circulates in said second part of the contactor.

15. The process of claim 1, wherein said circulating liquid has a temperature of between 5 and 10° C.

16. The process as claimed in claim 2, further comprising:
injecting the stale gas into a first part of the contactor, called the co-current part, in which the stale gas circulates in the same direction as the direction in which the liquid circulates in said first part of the contactor, and
circulating the stale gas in a second part of the contactor, called the counter-current part, in which the stale gas circulates in the direction opposite to the direction in which the liquid circulates in said second part of the contactor, and
circulating the stale gas in the filter unit.

17. The process as claimed in claim 3, further comprising:
injecting the stale gas into a first part of the contactor, called the co-current part, in which the stale gas circulates in the same direction as the direction in which the liquid circulates in said first part of the contactor, and
circulating the stale gas in a second part of the contactor, called the counter-current part, in which the stale gas circulates in the direction opposite to the direction in which the liquid circulates in said second part of the contactor, and
circulating the stale gas in the filter unit.

18. The process as claimed in claim 2, further comprising placing the stale gas and the liquid circulating in the contactor in contact with a heat exchanger.

19. The process as claimed in claim 3, further comprising placing the stale gas and the liquid circulating in the contactor in contact with a heat exchanger.

20. The process as claimed in claim 1, further comprising circulating a cooling liquid in the heat exchanger, said cooling liquid having a temperature of between 3 and 10° C.
